(19) United States
(12) Patent Application Publication
(10) Pub. No.: US 2006/0204571 A1
(43) Pub. Date: Sep. 14, 2006

(54) Stable compositions of bupropion or its pharmaceutically acceptable salts

(75) Inventors: Vaishali Vijay Dhavse, Mumbai (IN); Nitin Bhalachandra Dharmadhikari, Mumbai (IN)

Correspondence Address: MERCHANT & GOULD PC, P.O. BOX 2903, MINNEAPOLIS, MN, 55402-0903, US

(73) Assignee: Sun Pharmaceutical Industries Limited, Mumbai (IN)

(21) Appl. No.: 11/373,480

(22) Filed: Mar. 10, 2006

(30) Foreign Application Priority Data
Mar. 12, 2005 (IN) 240/MUM/2005

(51) Int. Cl. A61K 9/20; A61K 31/195

(52) U.S. Cl. 424/464; 514/567

(57) ABSTRACT

A stable oral pharmaceutical composition comprising a therapeutically effective amount of bupropion or its pharmaceutically acceptable salt intimately blending with one or more compatible excipients selected from the group consisting of talc and potassium chloride, additional pharmaceutically acceptable excipients and total impurities are present in amounts from 0% to not more than 3.3% w/w of the bupropion hydrochloride, when the composition is stored at 40° C. at 75% relative humidity for three months in closed containers with silica gel as dessicant.

### BACKGROUND

Bupropion hydrochloride, an antidepressant of the aminoketone class, is chemically unrelated to tricyclic, tetracyclic, or other known antidepressant agents. It is designated as (±)-1-(3-chlorophenyl)-2-[(1,1-dimethylethyl)amino]-1-propanone hydrochloride. It is a water-soluble, crystalline solid, highly hygroscopic and is susceptible to decomposition. Although bupropion hydrochloride is stable in bulk and in most simple blends, the drug is unstable in complex mixtures such as granulations or tablets.

U.S. Pat. Nos. 5,358,970; 5,541,231; 5,731,000 and 5,763,493 to Ruff et al describe a stabilized bupropion hydrochloride formulation having a stabilizer selected from group consisting of L-cysteine hydrochloride, glycine hydrochloride, malic acid, sodium metabisulfite, citric acid, tartaric acid, L-cystine dihydrochloride, ascorbic acid, and isoascorbic (erythorbic) acid. U.S. Pat. No. 6,652,882 to Odidi et. al describes stabilization of drug by a saturated polyglycolised glyceride like Gelucire®.

The other acid stabilization strategies of bupropion formulation are achieved by inorganic acids like hydrochloric acid, phosphoric acid, nitric acid and sulfuric acid (U.S. Pat. No. 5,968,553); dicarboxylic acids like oxalic acid, succinic acid, adipic acid, fumaric acid, benzoic acid and phthalic acid (U.S. Pat. Nos. 6,194,002; 6,221,917; 6,242,496; 6,482,987 and 6,652,882); sulfites like potassium metabisulfite and sodium bisulfite (U.S. Pat. No. 6,238,697); organic esters like L-ascorbic acid palmitate, tocopherol solution in alcohol, butylated hydroxy anisole, vitamin E succinate, vitamin E 700 acetate, and L-ascorbic acid G palmitate are used in transdermal preparations (U.S. Pat. No. 6,312,716). The use of acidified granules of microcrystalline cellulose (U.S. Pat. No. 6,153,223); salts of organic bases like creatinine hydrochloride, pyridoxine hydrochloride and thiamine hydrochloride and inorganic acid like potassium phosphate monobasic (U.S. Pat. No. 6,333,332) is also reported. The aforesaid prior arts require that the composition attains an acidic pH for stabilization of bupropion but such acidic pH may be undesirable for example, an added excipients susceptible to hydrolysis may degrade more rapidly at acidic pH as compared to neutral pH when the composition is stored on the shelf.

United States Patent Application No. 20050112198 discloses a pharmaceutical solid dosage form comprising bupropion hydrochloride and at least one member of the group consisting of butylated hydroxyanisole, butylated hyxroxytoluene and ion exchange resin.

U.S. Pat. No. 6,893,660 discloses a pharmaceutical composition in solid form comprising pharmaceutically active ingredients combined with excipients having a negative effect on stability of the active ingredients, by applying a sealing coating around the excipients having a negative effect. The invention requires a cumbersome and relatively expensive process of coating of the excipients. Depending on the excipients to be coated the process could be technically complex or difficult.

### SUMMARY

Accordingly, in an embodiment, the present invention provides a method of stabilization of oral pharmaceutical composition comprising therapeutically effective amounts of bupropion or its pharmaceutically acceptable salt.

Accordingly, in an embodiment, the present invention provides a stable oral pharmaceutical composition comprising bupropion or its pharmaceutically acceptable salt.

The present invention provides a method of stabilization of a pharmaceutical composition comprising a therapeutically effective amount of bupropion or its pharmaceutically acceptable salt. Upon blending bupropion or its pharmaceutically acceptable salt with one or more compatible excipients preferably talc and potassium chloride and additional pharmaceutically acceptable excipients, it was surprisingly found that the compositions so formed had total impurities in amounts from 0% by weight to not more than 3.3% by weight of bupropion hydrochloride, when the composition was stored at 40° C. at 75% relative humidity for 3 months in closed containers with silica gel as dessicant. It was a further surprising result to find that the composition was stable even if one or more the additional excipients had a destabilizing effect on bupropion or it's pharmaceutically acceptable salt. The same effect was not found if the compatible excipients such as talc and/or potassium chloride were not intimately blended but blended along with other excipients.

Thus present further provides a stable oral pharmaceutical composition comprising a therapeutically effective amount of bupropion or its pharmaceutically acceptable salt intimately blending with one or more compatible excipients selected preferably talc and/or potassium chloride, additional pharmaceutically acceptable excipients and total impurity which is present in amounts from 0% by weight to not more than 3.3% by weight of the bupropion hydrochloride when the composition is stored at 40° C. at 75% relative humidity for three months in closed containers with silica gel as dessicant.

### DETAILED DESCRIPTION

The present invention provides a method of stabilization of a pharmaceutical composition comprising a therapeutically effective amount of bupropion or its pharmaceutically acceptable salt. Upon blending bupropion or its pharmaceutically acceptable salt with preferably talc and/or potassium chloride and additional pharmaceutically acceptable excipients, the composition may be formed that have an total impurity in amounts from 0% by weight to not more than 3.3% by weight of bupropion hydrochloride, when the composition was stored at 40° C. at 75% relative humidity for three months in closed containers with silica gel as dessicant.

The bupropion or its pharmaceutically acceptable salt is used in a therapeutically effective amount in the stable oral pharmaceutical compositions of the present invention. The therapeutically effective amount of bupropion or its pharmaceutically acceptable salt that may be used in the stable composition of the present invention is in the range from about 70 mg to about 120 mg, preferably from about 75 mg to about 100 mg.

Talc is a hydrated magnesium sheet silicate with the chemical formula Mg3Si4O10(OH)2. The elementary sheet is composed of a layer of magnesium-oxygen/hydroxyl octahedra, sandwiched between two layers of silicon oxygen tetrahedra. The main or basal surfaces of this elementary sheet do not contain hydroxyl groups or active ions, which explains talc's hydrophobicity. It is thus proposed that upon blending talc forms a microenvironment around the particles of bupropion and due to it's hydrophobic nature talc reduces the availability of water on the surfaces of bupropion particles. It is also proposed that potassium chloride reduces the availability of water around the particles by solvating the water molecules, thereby masking the bupropion or its pharmaceutically acceptable salt from the water available. The compatible excipient is preferably a mixture of both talc and potassium chloride.

It was a further surprising result to find that the composition of the present invention was stable even if the additional excipients had a destabilizing effect on bupropion or it's pharmaceutically acceptable salt.

The additional pharmaceutical excipients that may be used according to the present invention are selected based on the drug excipients compatibility studies. Various excipients were intimately blended with bupropion or its pharmaceutically acceptable salt in the ratio ranging from about 0.1:1 to about 1:1 and stored at 60° C. for 15 days. The total impurities of the blend were determined by HPLC (Waters Symmetry, C-18, 4.6-mm×10-cm, 3.5-μ, thermostated to 25° C.; injection volume: 5 μL, flow rate: 1.5 ml per minute; 226 nm detection). The gradient elution used mobile phase of Acetonitrile:Trifluoroacetic acid:Methanol (57:23:20). If the total impurities, were less than 2% by weight of bupropion hydrochloride; the excipients were considered as compatible i.e without any destabilizing effect on bupropion or its pharmaceutically acceptable salts. The excipients which showed more than 2% total impurities, were categorized as excipients having destabilizing effect on bupropion. Accordingly ferrous fumarate, maltodextrin, alginic acid, crospovidone, lactose monohydrate, polyethylene glycol, methacrylates were found to be compatible whereas microcrystalline cellulose, pregelatinized starch, mannitol, stearic acid, hydroxypropyl cellulose, hydroxymethyl propyl cellulose were found to have total impurities more than 2% by weight of bupropion hydrochloride and therefore were categorized as excipients with destabilizing effect on bupropion.

According to one of the embodiments of the present invention, the composition is prepared by intimately blending bupropion or its pharmaceutically acceptable salt with talc and/or potassium chloride. Bupropion or its pharmaceutically acceptable salt is intimately blended with talc and/or potassium chloride in a double cone blender for a period of about 30 minutes.

The amount of talc that is used according to the present invention ranges from about 1% w/w to about 20% w/w of the total weight of the composition. Preferably the amount of talc that used ranges from 5% w/w to about 15% w/w of the total weight of the composition.

The amount of potassium chloride that may be used according to the present invention ranges from about 1% w/w to about 20% w/w of the total weight of the composition, preferably from about 3% w/w to about 15% w/w, most preferably 5% w/w to about 10% w/w of the total weight of the composition.

Preferably both talc and potassium chloride may be used. According to the present invention additional excipients that have a destabilizing effect on bupropion are not blended in the first stage of blending with talc, preferably also not in the second stage of blending with potassium chloride.

They may be blended with bupropion or its pharmaceutically acceptable salt in one stage i.e., together or in two stages i.e., first blend one and then the other. More preferably, a first intimate admixture of talc and bupropion or its pharmaceutically acceptable salt is prepared by blending and the admixture so obtained is further mixed with potassium chloride.

One of the preferred embodiments of the present invention uses ferrous fumarate as a additional excipients that has no destabilizing effect on bupropion or its pharmaceutically acceptable salt. The amount of ferrous fumarate that is used in the composition of the present invention, may range from about 1% to about 20% w/w of the composition, preferably from about 3% to 15% w/w of total weight of the composition, most preferably from about 5% w/w to about 10% w/w of total weight of the composition.

Another embodiments of the present invention uses maltodextrin as an additional excipients that has no destabilizing effect on bupropion or its pharmaceutically acceptable salt. The amount of maltodextrin that is used in the composition of the present invention, may range from about 1% w/w of the total weight of the composition to about 20% w/w of the total weight of the composition, preferably from about 3% to 15% w/w of the composition, most preferably from about 5% w/w to about 10% w/w of total weight of the composition.

In the preferred embodiments the addition of these additional excipients that have no destabilizing effect, is done after the initial intimate mixing of bupropion hydrochloride with talc and/or potassium chloride. When the composition of the present invention is prepared by wet granulation, these excipients may be added either intragranularly or extragranularly. When the composition is prepared by direct compression, these additional excipients may be added in the second stage of intimate blending of bupropion or its pharmaceutically acceptable salt with either talc or potassium chloride or both.

The present invention is advantageous in that the pre blend of bupropion or its pharmaceutically acceptable salt with talc and/or potassium chloride increases the options available to the formulator allowing the addition of the excipients having destabilizing effect on bupropion. When such excipients overcome some of the problems and/or help meet the other quality or manufacturing requirements. The present invention also overcomes the disadvantages of the prior art. The additional excipients added as fillers, binders, disintegrants and lubricants may thus either have a stabilizing or destabilizing effect on bupropion or its pharmaceutically acceptable salt.

In the more preferred embodiments of the present invention the composition is prepared by a dry procedure such as dry blending followed by filling the mixture into capsules or dry blending followed by dry granulation followed by compressing the granules into tablets or dry blending followed by compressing the mixture into tablets.

Fillers that may be used in the stable oral pharmaceutical composition of the present invention are selected from the group consisting of microcrystalline cellulose, mannitol, dextrates, dextrins, dextrose, fructose, lactose, lactitol, maltitol, maltodextrins, maltose and the like and mixtures thereof. Preferably the filler is microcrystalline cellulose. The commercially available grades of microcrystalline cellulose that may be used are Avicel PH-102 and Avicel PH-112, both having a mean particle size of 100 μm with moisture content being less than 5.0% and less than 1.5% respectively. Generally the amount of the lubricants used in the present invention may vary from about 0.001% to about 90% w/w of the composition.

The binders used in the present invention may be selected from the group comprising starch, gelatin, dextrin, maltodextrin, natural and synthetic gums like acacia, alginic acid, sodium alginate, guar gum, ghatti gum, carboxymethylcellulose, methylcellulose, hydroxyethyl cellulose, hydroxypropyl cellulose, hydroxypropyl methylcellulose, polyvinylpyrrolidone, veegum, arabogalactan and the like and mixtures thereof. Generally the amount of the binders used in the present invention may vary from about 0.5% w/w to about 10% w/w of the composition.

The lubricants used in the present invention may be selected from the group consisting of talc, magnesium stearate, calcium stearate, stearic acid, hydrogenated vegetable oils, polyethylene glycol and the like and mixtures thereof. Generally the amount of the lubricants used in the present invention may vary from about 0.001% to about 5% w/w of the composition.

Some more preferred compositions of the present invention are illustrated below

| Stages of | | Amount % w/w of |
|---|---|---|
| process | Ingredients | the composition |
| First blend | Bupropion hydrochloride | 10-40 |
| | talc | 5-15 |
| Second blend | Potassium chloride | 5-10 |
| Direct | Additional excipients that have no | 25-80 |
| compression | destabilizing effect on bupropion | |
| | Additional excipients that have a | 0-10 |
| | destabilizing effect on bupropion | |

The present invention may be better understood with reference to the following examples. These examples are intended to be representative of specific embodiments of the invention, and are not intended as limiting the scope of the invention.

### EXAMPLE 1

The oral pharmaceutical composition of the present invention was obtained as per the method given in Table 1 below.

TABLE 1

| | | % w/w of the |
|---|---|---|
| Stages of Process | Ingredients | composition |
| A. First Blend | Bupropion HCL | 23.1 |
| | Talc | 9.24 |
| B. Second Blend | Potassium chloride | 4.85 |
| C. Granulation with water | Pregelatinized Starch | 9.24 |
| | Lactose monohydrate | 28.20 |
| | crospovidone | 0.92 |
| D. Extragranular | Crospovidone | 0.92 |
| | Pregelatinized Starch | 2.77 |
| | Lactose anhydrous | 12.02 |
| | Talc | 4.85 |
| E. Coating | Opadry Coating | 2.91 |

Bupropion hydrochloride was blended in a double cone blender with talc for 20 min. Potassium chloride was added to this and blended further for 5 minutes. Lactose monohydrate, pregelatinized starch and crospovidone were further added and blended for 5 minutes, followed by granulation with water. The granules were dried, milled and blended with extragranular materials and compressed into tablets. These lubricated granules were compressed into tablets. The core tablets were coated with 12% w/w dispersion of Opadry red 03B55304 (HPMC based) in water in a perforated coating pan. The tablets were stored at 40° C. at 75% relative humidity in closed containers with silica gel as dessicant. The total impurities were found to be 0.81% by weight of bupropion hydrochloride.

### EXAMPLE 2

TABLE 2

| | | % w/w of the |
|---|---|---|
| Stages of Process | Ingredients | composition |
| A. First Blend | Bupropion HCL | 35.56 |
| | Talc | 7.1 |
| B. Second Blend | Potassium chloride | 5.51 |
| C. Granulation with water | Pregelatinized Starch | 7.4 |
| | Crospovidone | 0.71 |
| | Lactose monohydrate | 21.70 |
| D. Extragranular excipients | Lactose anhydrous | 10.66 |
| | Crospovidone | 0.71 |
| | Talc | 6.93 |
| E. Coating | Opadry Red 03 B55304 | 2.91 |

Bupropion hydrochloride was blended in a double cone blender with talc for 20 min. Potassium chloride was added to this and further blended for about 10 minutes. Lactose monohydrate and pregelatinized starch were further added and blended for 5 minutes, followed by granulation with water. The granules were dried, milled and blended with extragranular materials and compressed into tablets. These lubricated granules were compressed into tablets. The core tablets were coated with 12% w/w dispersion of Opadry rf 03b55304 (HPMC based) in water in a perforated coating pan. The tablets were stored at 40° C. at 75% relative humidity in closed containers with silica gel as dessicant. The total impurities were found to be 0.65% by weight of bupropion hydrochloride.

### EXAMPLE 3

TABLE 3

| | | % w/w of the |
|---|---|---|
| Stages of Process | Ingredients | composition |
| A. First Blend | Bupropion HCL | 23.1 |
| | Talc | 9.24 |
| B. Second Blend | Potassium chloride | 9.52 |
| C. Granulation with water | maltodextrin | 4.85 |
| | Pregelatinized Starch | 4.85 |
| | Lactose monohydrate | 19.6 |
| | Ferrous fumarate | 4.62 |
| D. Extragranular | Crospovidone | 1.84 |
| | Talc | 5.77 |
| | Lactose anhydrous | 13.86 |
| E. Coating | Opadry Red 03 B55304 | 2.91 |

Bupropion hydrochloride was blended with talc in a double cone blender for 20 minutes. Potassium chloride was sieved through 80 mesh and blended with the blend of bupropion hydrochloride and talc. Lactose monohydrate, prelgelatinized starch, maltodextrin and ferrous fumarate were sifted through 40 mesh individually and the blend of bupropion hydrochloride with talc and potassium chloride were dry mixed and granulated with water. The granules were dried, milled and blended with the extragranular excipients namely crospovidone, lactose anhydrous and talc. These lubricated granules were compressed into tablets. The core tablets were coated with 12% w/w dispersion of Opadry 03b55304 (HPMC based) in water in a perforated coating pan. The tablets were stored at 40° C. at 75% relative humidity in closed containers with silica gel as dessicant. The total impurities were found to be 0.47% by weight of bupropion hydrochloride.

### EXAMPLE 4

TABLE 4

| | | % w/w |
|---|---|---|
| Stages of | | of the |
| Process | Ingredients | composition |
| A. First Blend | Bupropion HCL | 23.1 |
| | Talc | 9.24 |
| B. Second Blend | Potassium chloride | 9.52 |
| C. Direct compression | maltodextrin | 4.85 |
| | Pregelatinized Starch | 7.85 |
| | Lactose monohydrate (Pharmatose | 35.36 |
| | DCL 11) | |
| | crospovidone | 1.84 |
| | Ferrous fumarate | 4.62 |
| | Talc | 5.77 |
| E. Coating | Opadry Red 03 B55304 | 2.91 |

Bupropion hydrochloride and talc were sifted through 40 and 80 mesh respectively and blended for 20 minutes in a double cone blender. Powder grade potassium chloride was sifted through 80 mesh and added to the double cone blender and blended further for 5 minutes. Lactose monohydrate, pregelatinized starch, maltodextrin, ferrous fumarate and crospovidone were sifted through 40 mesh and mixed with the blend of bupropion hydrochloride. Talc was sifted and added to the blend. The total blend was blended in a double cone blender for 30 minutes. The blend as directly compressed into tablet cores. The core tablets were coated with 12% w/w dispersion of Opadry red 03b55304 (HPMC based) in water in a perforated coating pan. The tablets were stored at 40° C. at 75% relative humidity in closed containers with silica gel as dessicant. The total impurities were found to be 0.49% by weight of bupropion hydrochloride.

### EXAMPLE 5

TABLE 5

| Stages | Ingredients | % w/w of the tablet |
|---|---|---|
| First blend | Bupropion hydrochloride | 15.7 |
| | Potassium chloride | 4.88 |
| Second blend | Lactose anhydrous | 24.9 |
| | Pregelatinized starch | 32.78 |
| Granulation with | Water | q.s |
| water | | |
| extragranular | crospovidone | 1.89 |
| | Lactose anhydrous | 8.9 |
| | talc | 7.88 |
| Coating | Opadry red O3B55304 | 2.91 |

Bupropion hydrochloride and potassium chloride were dry mixed. Lactose anhydrous and pregelatinized starch were added to the mix. The mixture was granulated with water, dried to obtain granules. Milled granules were blended with extragranular excipients namely crospovidone and lactose anhydrous. Talc was added as a lubricant. The blend was compressed into tablets. The tablet cores were coated with Opadry red (HPMC based). The core tablets were coated with 12% w/w dispersion of Opadry red 03b55304 (HPMC based) in water in a perforated coating pan. The tablets were stored at 40° C. at 75% relative humidity in closed containers with silica gel as dessicant. The total impurities were found to be 2.22% by weight of bupropion hydrochloride.

### COMPARATIVE EXAMPLE 1

TABLE 6

| Ingredients | % w/w of the tablet |
|---|---|
| Bupropion hydrochloride | 15.75 |
| Microcrystalline cellulose | 63.20 |
| Hydroxypropyl Cellulose | 9.940 |
| L-HPC LH-21 | 1.97 |
| Talc | q.s for lubrication |
| Microcrystalline cellulose | 1.97 |
| Opadry yellow | ˜3 |

Bupropion hydrochloride, microcrystalline cellulose and hydroxypropyl cellulose were sifted, mixed together and granulated with water. The granules were dry milled. The dry granules were further blended with microcrystalline cellulose and hydroxypropyl cellulose. The granules were lubricated with talc and compressed into tablets. The compressed tablets were coated with aqueous Opadry yellow. The tablets were stored at 40° C. at 75% relative humidity in closed containers with silica gel as dessicant. The total impurities were found to be 27.75% by weight of bupropion hydrochloride.

### COMPARATIVE EXAMPLE 2

TABLE 7

| | % w/w of the |
|---|---|
| Ingredients | tablet |
| Bupropion hydrochloride | 15.75 |
| Lactose monohydrate | 63.2 |
| Hydroxypropyl Cellulose | 13.39 |
| Talc | q.s |
| Stearic acid | 1.89 |
| Opadry yellow | ˜3 |

Bupropion hydrochloride, lactose and hydroxypropyl cellulose were sifted, mixed together and granulated with water. The granules were dry milled. The dry granules were further blended with hydroxypropyl cellulose. The granules were lubricated with mixture of stearic acid and talc and compressed into tablets. The compressed tablets were coated with aqueous Opadry yellow. The tablets were stored at 40° C. at 75% relative humidity in closed containers with silica gel as dessicant. The total impurities were found to be 7.31% by weight of bupropion hydrochloride.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

All publications and patent applications in this specification are indicative of the level of ordinary skill in the art to which this invention pertains.

The invention has been described with reference to various specific and preferred embodiments and techniques. However, it should be understood that many variations and modifications may be made while remaining within the spirit and scope of the invention.

We claim:

1. A stable oral pharmaceutical composition comprising
(a) a therapeutically effective amount of bupropion or its pharmaceutically acceptable salt intimately blending with one or more compatible excipients selected from the group consisting of talc and potassium chloride,
(b) additional pharmaceutically acceptable excipients and
(c) total impurities present in amounts from 0% to not more than 3.3% w/w of the bupropion hydrochloride when the composition is stored at 40° C. at 75% relative humidity for three months in closed containers with silica gel as dessicant.
(d)

2. A stable oral pharmaceutical composition in claim 1 wherein additional pharmaceutically acceptable excipents are selected from excipients that do not have a destabilizing effect on bupropion.

3. A stable oral pharmaceutical composition in claim 2 wherein the additional pharmaceutically acceptable excipents are selected from the group consisting of ferrous fumarate, maltodextrin, methacrylate, polyethylene glycol, lactose monohydrate, methacrylates, alginic acid, crospovidone and mixtures thereof.

4. A stable oral pharmaceutical composition in claim 1 wherein additional excipients are selected from excipients having a destabilizing effect on bupropion.

5. A stable oral pharmaceutical composition in claim 4 wherein the additional pharmaceutically acceptable excipients are selected from the group consisting pregelatinized starch, microcrystalline cellulose, pregelatinized starch, mannitol, stearic acid, hydroxypropyl cellulose, hydroxymethyl propyl cellulose.

6. A stable oral pharmaceutical composition as claimed in claim 1 wherein the compatible excipient is talc present in an amount ranging from about 5% to about 15% by weight of the composition.

7. A stable oral pharmaceutical composition in claim 1 wherein compatible excipient is potassium chloride present in amounts ranging from about 5% w/w to about 10% w/w of the total weight composition.

8. A stable oral pharmaceutical composition in claim 3 wherein excipient is ferrous fumarate in an that ranges from about 5% to about 10% by weight of the composition.

9. A stable oral pharmaceutical composition in claim 2 wherein the amount of maltodextrin ranges from about 3% to about 10% by weight of the composition.

10. A stable oral pharmaceutical composition comprising
a) bupropion or its pharmaceutically acceptable salt in amounts in the range from about 10% to 50% by weight of the composition intimately blended with talc in amounts in the range from about 5% to 15% by weight of the composition
b) potassium chloride blended with mixture of bupropion or its pharmaceutically acceptable salt and talc in amounts ranging from about 5% to about 10% of the weight of the composition
c) additional pharmaceutically acceptable excipients comprising
i) excipients having no destabilizing effect on bupropion or its pharmaceutically acceptable salt in amount ranging from about 25% to about 80% by weight of the composition
ii) excipients having destabilizing effect on bupropion or its pharmaceutically acceptable salt in amount ranging from about 0% to about 10% by weight of the composition.
